(19) United States
(12) Patent Application Publication
(10) Pub. No.: US 2024/0288396 A1
(43) Pub. Date: Aug. 29, 2024

(54) BIOSENSOR

(71) Applicant: DONGWOO FINE-CHEM CO., LTD., Jeollabuk-do (KR)

(72) Inventors: Dong-Yeop LEE, Pyeongtaek-si (KR); Dong-Ok KIM, Pyeongtaek-si (KR)

(21) Appl. No.: 18/572,068

(22) PCT Filed: Jun. 28, 2022
(86) PCT No.: PCT/KR2022/009217

§ 371 Date: Dec. 19, 2023

(30) Foreign Application Priority Data
Jul. 1, 2021 (KR) 10-2021-0086337

(51) Int. Cl. G01N 27/327; G01N 27/30

(52) U.S. Cl. CPC G01N 27/3271; G01N 27/301

(57) ABSTRACT

The present disclosure relates to a biosensor including a first substrate having a hydrophobic surface on one side thereof, a second substrate disposed on the first substrate and having a hydrophilic surface on one side thereof, a support layer having a predetermined height to provide a space between the hydrophobic surface of the first substrate and the hydrophilic surface of the second substrate, one or more electrode layers formed in the space, and an enzyme reaction layer formed on the electrode layer.

### TECHNICAL FIELD

The present disclosure relates to a biosensor.

### BACKGROUND ART

A biosensor is a device or element that can determine the presence or amount of an analyte being tested in a manner that a bio-receptor with selective specificity thereof reacts with the analyte and a signal transducer thereof measures the extent of the reaction.

Such biosensors are classified into electrochemical sensors, thermal sensors, optical sensors, etc. according to the conversion methods. Recently, depending on the type of target substance to be analyzed, the biosensors are diversely named, for example, glucose sensors, cell sensors, immune biosensors, DNA chips, etc.

Among them, the electrochemical sensor has been widely used as a biosensor conversion method in terms of being able to convert the amount of a biological sample into an electrical signal that can be easily processed into information.

Korean Patent Application Publication No. 10-2004-0105429 relates to an electrochemical biosensor using blood as a sample and discloses a blood glucose sensor capable of reducing a measurement error depending on a hematocrit level.

However, compared to a blood glucose sensor using blood, non-invasive measurement of glucose concentration using bodily fluids such as saliva, sweat, and tears requires a large amount of sample. However, since the amount of body fluid is limited and the body fluid contains a very small amount of glucose, it is difficult to measure the glucose concentration from the body fluid.

In order to accurately measure the sugar concentration in a small amount of body fluid, it is necessary to improve the resolution of the sensor. To improve the body fluid analysis resolution, a large amount of enzyme must be applied, or expensive electrode materials must be used. However, the application amount of the enzyme is limited, and mass production of such a sensor is difficult. Accordingly, there is a need to develop biosensors that are low-cost, simple to fabricate, and have an improved body fluid analysis resolution.

### DISCLOSURE

### Technical Problem

The present disclosure is to solve the problems of the related art described above and aims to provide a biosensor capable of measuring glucose even with a very small amount of bodily fluid.

Specifically, in order to solve the problem that existing non-invasive glucose sensors using body fluids require a larger amount of sample than blood glucose sensors, the present disclosure provides a biosensor with improved analysis resolution and improved concentration measurement accuracy even while using a very small amount of body fluid.

The objectives to be achieved by the present disclosure are not limited to the ones mentioned above, and other objectives not mentioned above can be clearly understood by those skilled in the art from the following description.

### Technical Solution

The present disclosure provides a biosensor including a first substrate having a hydrophobic surface on one side thereof, a second substrate disposed on the first substrate and having a hydrophilic surface on one side thereof, a support layer having a predetermined height to provide a space between the hydrophobic surface of the first substrate and the hydrophilic surface of the second substrate, one or more electrode layers formed in the space, and an enzyme reaction layer formed on the electrode layer.

According to one embodiment of the present disclosure, the support layer may have a height (y) of greater than about 25 μm and less than about 300 μm, and may be made of a polymer resin.

According to one embodiment of the present disclosure, the enzyme reaction layer may include one or more selected from the group consisting of oxidases and dehydrogenases. For example, the oxidases may include at least one selected from the group consisting of cholesterol oxidase, lactate oxidase, ascorbic acid oxidase, and alcohol oxidase, and the dehydrogenation, and the dehydrogenases may include at least one selected from the group consisting of glucose dehydrogenase, glutamate dehydrogenase, lactate dehydrogenase, and alcohol dehydrogenase.

According to one embodiment of the present disclosure, the electrode layer may include a working electrode layer and a reference electrode layer. The electrode layer may include at least one selected from the group consisting of a carbon electrode layer and a metal electrode layer. For example, the metal electrode layer may include a metal layer and a metal protective layer formed on the metal layer.

According to one embodiment of the present disclosure, the enzyme reaction layer may be disposed on the working electrode layer, and may have an area that is 60% to 330% of the area of the working electrode layer.

### Advantageous Effects

The biosensor of the present disclosure can accurately measure glucose even from a very small amount of body fluid. Specifically, the biosensor of the present disclosure improves resolution by adjusting the height of the support layer and the width of the enzyme reaction layer in a structure in which an empty space is defined by disposing a cover composed of the support layer and an upper substrate on the enzyme reaction layer. Therefore, the biosensor of the present disclosure provides the effect of measuring a finer concentration from a very small amount of body fluid (sample).

### DESCRIPTION OF DRAWINGS

FIG. 1 is an exploded perspective view of a capillary-type biosensor according to an embodiment of the present disclosure;

FIG. 2 is a cross-sectional view taken along line B-B′ in FIG. 1;

FIG. 3 is a top view of a capillary-type biosensor according to an embodiment of the present disclosure (first substrate and second substrate are not shown);

FIG. 4 is an exploded perspective view of a microfluidics-type biosensor according to an embodiment of the present disclosure;

FIG. 5 is a cross-sectional view taken along line B-B′ in FIG. 4;

FIG. 6 is a top view of a microfluidics-type biosensor according to an embodiment of the present disclosure (first substrate and second substrate are not shown);

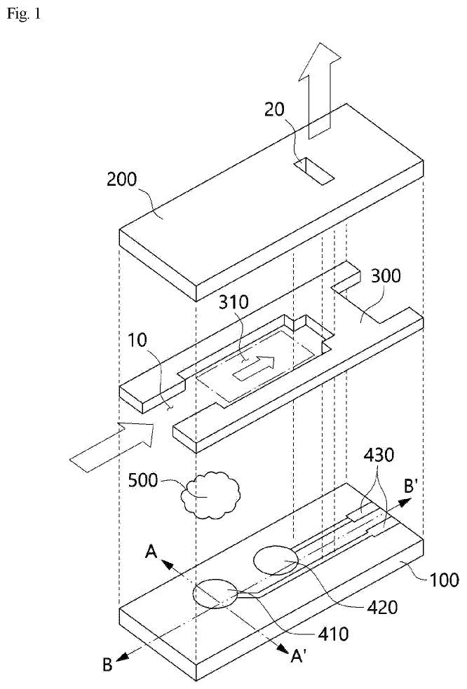

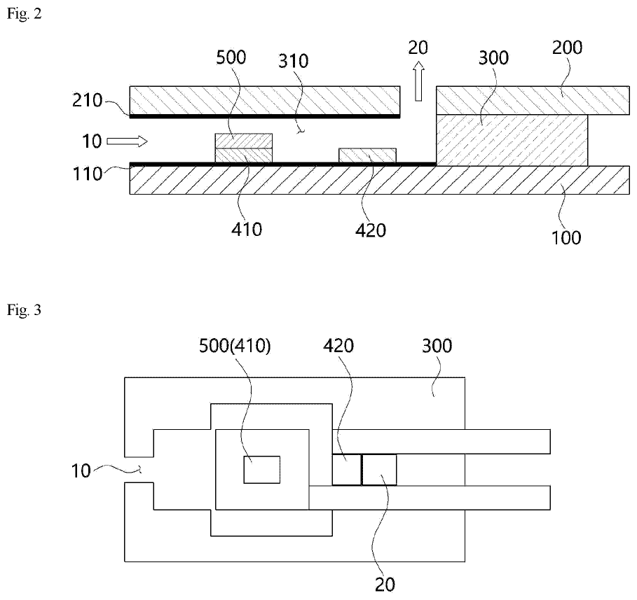

FIG. 2 is a cross-sectional view taken along line A-A′ in FIG. 1;

FIG. 8 is a cross-sectional view schematically illustrating the structure of a reduced type, a basic type, and an expanded type according to an exemplary embodiment of the present application;

FIG. 9 is a graph showing the evaluation results of Table 3 in an experimental example of the present application;

FIG. 10 is a graph showing changes in slope according to the height (x) of a support layer and the area (y) of an enzyme reaction layer in an experimental example of the present application; and

FIG. 11 is a graph showing changes in slope according to the height (x) of a support layer and the volume of a sample (solution) in an experimental example of the present application.

Reference signs in the drawings are as follows:

| | |
|---|---|
| 100: First substrate | 200: Second substrate |
| 110: Hydrophobic surface | 210: Hydrophilic surface |
| 300: Support layer | 310: Space (empty space) |
| 400: Electrode layer | 500: Enzyme reaction layer |

### BEST MODE

The present disclosure relates to a biosensor for improving the detection accuracy and precision of a sensor even with a very small amount of body fluid by improving analysis resolution by adjusting the height of a support layer and the area of an enzyme reaction layer.

Specifically, the present disclosure relates to a biosensor including a first substrate having a hydrophobic surface on one side thereof, a second substrate disposed on the first substrate and having a hydrophilic surface on one side thereof, a support layer having a predetermined height to provide a space between the hydrophobic surface of the first substrate and the hydrophilic surface of the second substrate, one or more electrode layers formed in the space, and an enzyme reaction layer formed on the electrode layer.

In the biosensor of the present disclosure, a test sample may be a biological sample such as blood, body fluid (saliva, sweat, tears, etc.), urine, or other liquid, but most preferably, the test sample is a body fluid (saliva, sweat, tears, etc.). For example, the test sample may contain glucose or lactic acid (lactate).

In order to accurately measure the glucose concentration in a small amount of body fluid, it is necessary to improve the analysis resolution of a sensor. To improve the analysis resolution, it is general to apply a large amount of enzyme to a sensor or to use an expensive electrode material in the sensor. However, the conventional methods are not suitable for mass production of sensors. Therefore, it is necessary to use a cover that can be easily manufactured at low cost, and the analysis resolution can be improved according to the construction of the cover even the same amount of enzyme is applied. For example, the biosensor of the present disclosure uses a cover composed of a support layer and a second substrate (upper substrate). The biosensor can measure the glucose concentration even from a small amount (less than 10 μL) of a sample (body fluid). Preferably, the biosensor can measure the glucose concentration from a sample (body fluid) of 1 to 7 μL. When the amount of the sample satisfies the above range, the glucose concentration can be accurately measured even with a small amount of body fluid.

Hereinafter, embodiments of the present disclosure will be described in detail with reference to the drawings. The drawings attached to the present specification illustrate preferred embodiments of the present disclosure and serve to aid understanding of the spirit of the present disclosure in conjunction with the description herein. Therefore, the present disclosure should not be construed as being limited to those illustrated in the drawings.

The terminology used herein is for describing the embodiments and is not intended to limit the scope of the present disclosure. As used herein, the singular forms “a”, “an”, and “the” are intended to include the plural forms as well, unless the context clearly indicates otherwise.

It will be further understood that the terms “comprise” and/or “comprising” when used in this specification specify the presence of stated features, regions, integers, steps, operations, elements and/or components, but do not preclude the presence or addition of one or more other features, regions, integers, steps, operations, elements, components and/or groups thereof. Like reference signs refer to like elements throughout the description herein.

The spatially relative terms “below”, “under”, “above”, “on”, and the like may be used used herein to easily describe the correlation of a device or element and another device or element as illustrated in the figures. The spatially relative terms should be understood as the terms that include different orientations of devices or elements being in other uses or operation in addition to the orientations illustrated in the figures. For example, when one device illustrated in the figures is turned over, the device described as disposed “below” or “under” another device element may be disposed “above” or “on” the other device. Accordingly, the exemplary term “below” may include orientations of both below and above. The device may be oriented at other orientations, and the spatially relative terms used herein may be interpreted accordingly.

### <Biosensor>

A biosensor according to the present disclosure includes: a first substrate 100 having a hydrophobic surface 110 on one side thereof; a second substrate 200 disposed on the first substrate 100 and having a hydrophilic surface 210 on one side thereof; a support layer 300 having a predetermined height to provide a space 310 between the hydrophobic surface 110 of the first substrate 100 and the hydrophilic surface 210 of the second substrate 200; one or more electrode layers 400 disposed in the space 310; and an enzyme reaction layer 500 disposed on the electrode layer 400.

The space 310 can adjust the amount of the sample coming into the space 310 according to the design of each component and the height of the support layer 300. Therefore, when the sample is injected, the amount of the sample can be adjusted by changing the area of the space 310 by changing the configuration design under the condition that the flowing of the sample is not interfered, and the height of the support layer 300 is selected to determine the amount of the sample. When the height of the support layer 300 is increased, the height of the space is increased, and thus the amount of sample to be injected can be increased. Therefore, the analysis resolution also increases as the height of the support layer 300 increases. In the present application, the height (thickness) of the space means the distance between the upper surface of the first substrate and the lower surface of the second substrate, and may be the same as the height (thickness) of the support laver.

In addition, the resolution (slope) may increase when the area of the enzyme reaction layer 500 is increased within the allowable range, but the resolution (slope) is not affected when the amount of the sample is adjusted. This can be explained by difference between the reaction area of the enzyme reaction layer 500 and the diffusion caused by the convection of the sample. A large increase in the amount of the sample applied on the enzyme reaction layer 500 means that the length from the upper surface of the sample to the upper surface of the reactant increases. However, an increase in the thickness of the enzyme reaction layer 500 does not mean an increase in the extent of reaction. To achieve an unimpeded reaction at an appropriate thickness, after a primary reaction occurs on the upper surface of the enzyme reaction layer 500, convection needs to occur in the sample so that the reacted portion of the sample and the unreacted portion of the sample change their position so that a secondary reaction with the unreacted portion of the sample on the enzyme reaction layer 500 can be facilitated. This may increase the measurement value. However, since it takes a longer time to induce convection as the thickness of the enzyme reaction layer 500 increases, the measurement values may soon converge on the extension line of the thickness increase curve.

Therefore, the height of the space 310 in which the electrode layer 400 is to be formed is suitably 1 μm to 500 μm, and preferably 10 μm to 300 μm in terms of the convenience of the process operation, and more preferable 50 μm to 300 μm in terms that a flexible patch type is employed and the space is used as an extended flow channel. It is preferable that the thickness of the channel extending from the electrode layer 400, that is, the thickness of the sample inlet 10, is preferably larger than the height of the space 310 in which the electrode layer 400 is formed, for the unimpeded flow of the solution.

The biosensor according to the present disclosure may theoretically have a relational expression such as Expression 1 below to facilitate measurement of a body fluid. In terms of accuracy and efficiency of measurement, the Z value of Expression 1 below represents the analysis resolution (slope). The Z value preferably falls within a range of 30 to 700 nA·mM⁻¹ and more preferably a range of 60 to 700 nA·mM⁻¹.

$$Z = γ + α x + β y$$

In Expression 1, Z=slope (nA·mM⁻¹), x=height (mm) of the support layer, y=area (mm²) of the enzyme reaction layer, γ=−147 (nA·mM⁻¹), α=2828 (nA·mM⁻¹·mm⁻¹), and β=13.9 (nA·mM⁻¹·mm⁻²).

However, the height (y) of the support layer needs to be thick enough to allow the sample to pass through the space. At an excessively large or small thickness, the pressure of capillary action acts, or the flow rate of the solution may fluctuate. Therefore, the excessively large or small thickness is not suitable for application to Expression 1. The area (x) of the enzyme reaction layer is preferably identical to the entire area of the working electrode layer in terms of estimating the slope (analysis resolution) using the expression.

FIG. 1 is an exploded perspective view of a capillary-type biosensor according to an embodiment of the present disclosure, FIG. 2 is a cross-sectional view taken along line B-B′ of FIG. 1, and FIG. 3 is a top view of a capillary-type biosensor according to an embodiment of the present disclosure (a first substrate and a second substrate are not shown).

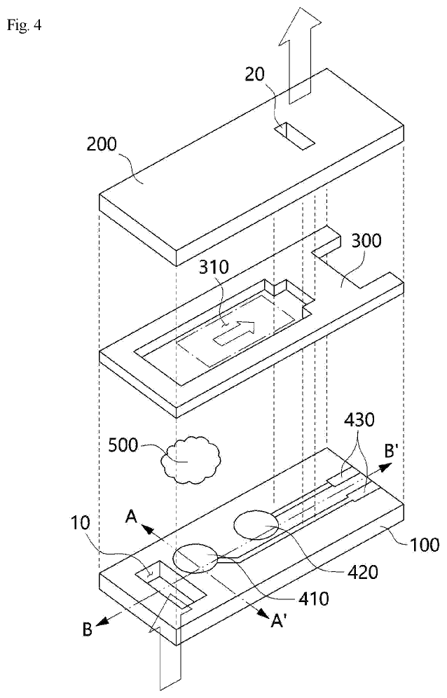

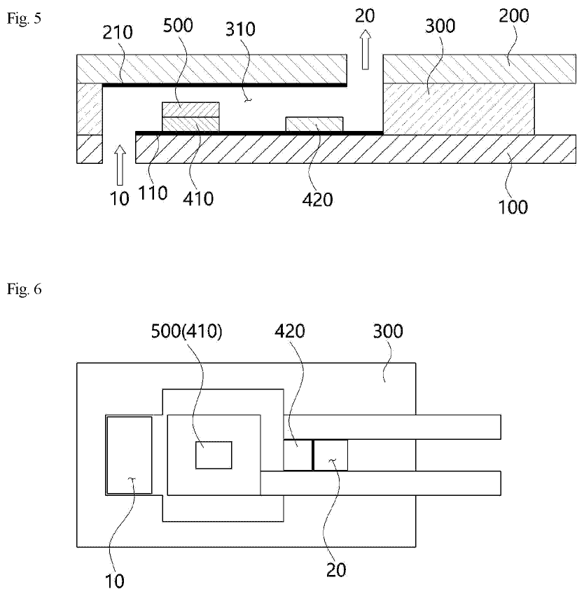

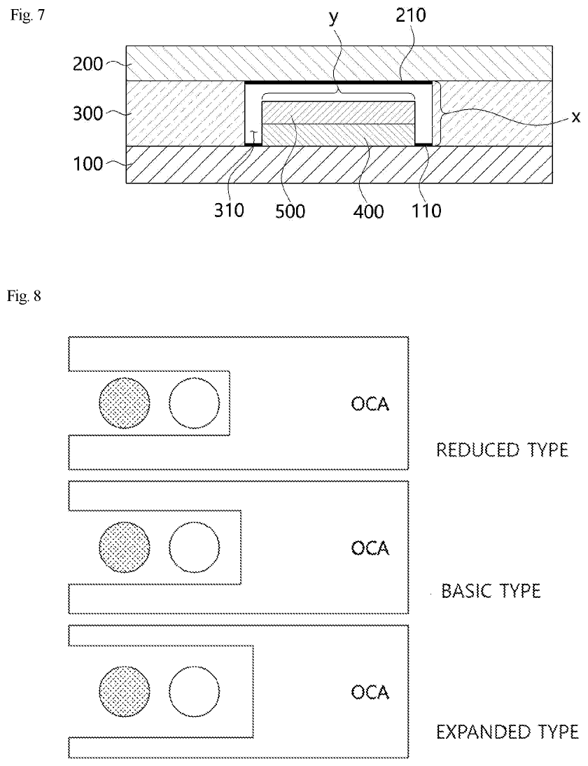

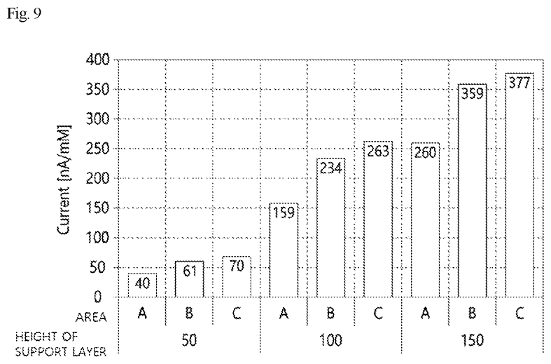

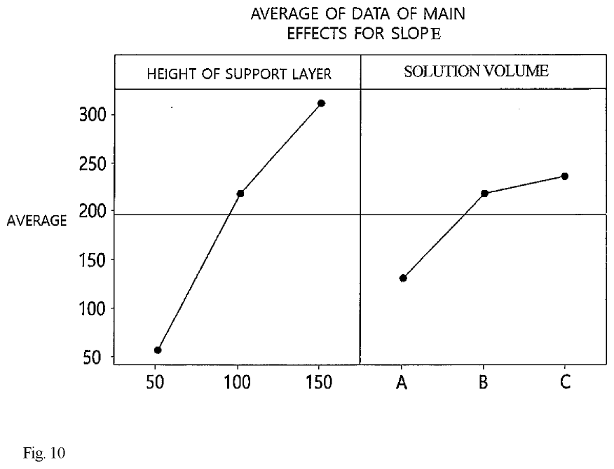

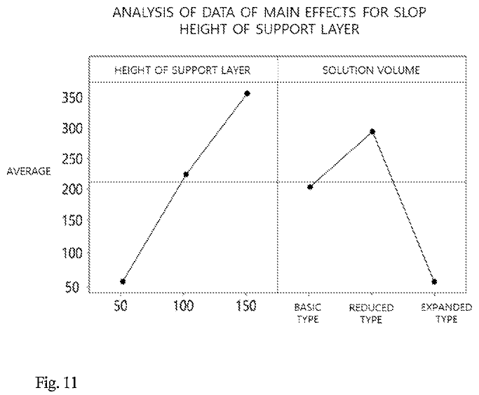

FIG. 4 is an exploded perspective view of a microfluidics-type biosensor according to an embodiment of the present disclosure, FIG. 5 is a cross-sectional view taken along line B-B′ of FIG. 4, and FIG. 6 is a top view of a microfluidics-type biosensor according to an embodiment of the present disclosure (a first substrate and a second substrate are not shown).

FIG. 7 is a cross-sectional view taken along lines A-A′ of FIGS. 1 and 4.

Referring to FIGS. 1 to 7, a biosensor according to an embodiment of the present disclosure includes a first substrate 100, a second substrate 200, a support layer 300, a space 310, an electrode layer 400, and an enzyme reaction layer 500. The electrode layer 400 may be composed of a working electrode layer 410 and a reference electrode layer 420, and the enzyme reaction layer 500 may be formed on the working electrode layer 410.

The first substrate 100 has a hydrophobic surface and serves to provide a structural base for the components constituting the biosensor. The second substrate 200 has a hydrophilic surface and serves to provide a structural cover for covering the components constituting the biosensor in conjunction with the support layer 300. In the present disclosure, the structure in which the first substrate has a hydrophobic surface, and the second substrate has a hydrophilic surface may mean that the first substrate and the second substrate have relative hydrophobicity or hydrophilicity to each other.

The first substrate 100 and the second substrate 200 may each independently be made of a hard material such as glass or be implemented in the form of a film having a flexible property. Existing products or novel products to be developed in the future may be used as the first and second substrates 100 and 200.

In one or more embodiments, the first substrate 100 and the second substrate 200 may be made of a hard material such as silicon, glass, glass epoxy, or ceramic. Alternatively, the first substrate 100 and the second substrate 200 may include a flexible film made of any one selected from: polyester resins such as polyethylene terephthalate (PET), polyethylene isophthalate, polyethylene naphthalate, and poly butylene terephthalate; cellulosic resins such as diacetyl cellulose and triacetyl cellulose; polycarbonate-based resins; acrylic resins such as polymethyl (meth)acrylate and polyethyl (meth)acrylate; styrenic resins such as polystyrene and acrylonitrile-styrene copolymer; polyolefin-based resins such as polyethylene, polypropylene, polyolefins having a cyclo-based or norbornene structure, and ethylene-propylene copolymers; vinyl chloride-based resins; amide resins such as nylon and aromatic polyamide; imide-based resins; polyethersulfone-based resins; sulfone-based resins; polyether ether ketone-based resins; sulfurized polyphenylene-based resins; vinyl alcohol-based resin; vinylidene chloride-based resins; vinyl butyral-based resins; allylate-based resins; polyoxymethylene-based resin; thermoplastic resins such as epoxy resins; and combinations thereof. In addition, the first and second substrates may include a film made of a thermosetting resin or an ultraviolet curable resin such as (meth)acrylic, urethane, acrylurethane, epoxy, or silicone, but the material of the film is not limited thereto. The thickness of the first substrate 100 and/or the second substrate 200 is not particularly limited, but may be 1 to 500 μm, preferably 1 to 300 μm, and more preferably 5 to 200 μm in terms of strength, handling, workability, and thin layer properties.

In some embodiments, the first substrate 100 and/or the second substrate 200 may contain additives. Examples of the additive include an ultraviolet absorber, an antioxidant, a lubricant, a plasticizer, a releasing agent, an anti-coloring agent, a flame retardant, a nucleating agent, an antistatic agent, a pigment, a colorant, and the like.

In some embodiments, the first substrate 100 and/or the second substrate 200 may include a functional layer on at least one surface thereof. The functional layer may have a structure including various functional layers such as a hard coating layer, an antireflection layer, and a gas barrier layer, and the functional layer is not limited to the examples. Various functional layers are used depending on the usage of the substrates.

In addition, the first substrate 100 and/or the second substrate 200 may be surface treated. Specifically, preferably, at least one surface (for example, upper surface) of the first substrate 100 is treated to have the hydrophobic surface 110, and at least one surface (for example, lower surface) of the second substrate 200 is treated to have the hydrophilic surface 210. For example, the surface treatment may be performed by dry treatment such as plasma treatment, corona treatment, and primer treatment, or chemical treatment such as alkali treatment including saponification.

In one embodiment of the present disclosure, in order to induce an unimpeded flow using the capillary effect in the space 310 in the structure shown in FIG. 1, it is preferable that the upper surface of the first substrate 100 is surface-treated to have hydrophobicity and the lower surface of the second substrate 200 is surface-treated to have hydrophilicity. In addition, since the enzyme reaction layer 500 is hydrophilic, it is preferable to ensure that the hydrophobic superficial layer of the first substrate has a thickness of at least 50 μm or more in order to facilitate the unimpeded flow of the sample.

In one embodiment of the present disclosure, in the case of the microfluidics-type simulated structure as shown in FIG. 4, it is preferable that both the lower and upper surfaces of the first substrate 100 are subjected to hydrophobic surface treatment, and the lower surface of the second substrate 200 is preferably subjected to hydrophilic surface treatment.

In some embodiments, the first substrate 100 may include a sample inlet 10, which is an opening for inserting a sample, as shown in FIG. 4. In addition, the second substrate 200 may include a gas outlet 20 that is an opening for discharging internal air to cause a capillary effect on the sample, as shown in FIGS. 1 and 4.

In some embodiments, the sample injected into the biosensor through the sample inlet 10 comes into contact with the enzyme reaction layer 500 while passing through the space 310, and the internal air is purged to the outside through the gas outlet 20.

The support layer 300 is a component that serves as a cover in conjunction with the second substrate 200. The support layer 300 has a predetermined thickness and is disposed on the first substrate 100 to support the second substrate 200. In the structure, an empty zone in which the support layer 300 is not yet formed is provided between the first substrate 100 and the second substrate 200 and is referred to as the space 310.

In one embodiment, the support layer 300 may be formed in contact with the upper surface of the first substrate 100, or may be formed to be adjacent to side surfaces of the electrode layer 400 and the enzyme reaction layer 500. For example, the support layer 300 may be configured to entirely expose the upper surfaces of the electrode layer 400 and the enzyme reaction layer 500.

In one embodiment, the support layer 300 may be formed to be adjacent to the upper and side surfaces of a wiring unit 430. For example, it may be disposed in a form of covering part or all of the wires 430.

In one embodiment, the support layer 300 may be formed to have a height that is greater than about 25 μm and is equal to or less than about 500 μm, and is preferably formed to have a height of 50 to 300 μm. When the height range is satisfied, the analysis resolution can be improved while minimizing the sample amount used.

In one embodiment, the support layer 300 may be made of a polymer resin. The type of polymer resin is not particularly limited. The polymer resin not limitedly includes at least one selected from the group consisting of optically clear adhesive (OCA), pressure sensitive adhesive (PSA), optically clear resin (OCR), polyacrylate, polymethacry late (for example, PMMA), polyimide, polyamide, polyvinyl alcohol, polyamic acid, polyolefin (for example, PE or PP), polystyrene, polynorbomene, phenylmaleimide copolymer, polyazobenzene, polyphenylenephthalamide, polyester (for example, PET or PBT), polyarylate, cinnamate-based polymer, coumarin-based polymer, phthalimidine-based polymer, chalcone-based polymer, and aromatic acetylene-based polymer.

In one embodiment, the amount of the sample injected into the space can be adjusted according to the height of the support layer 300 and the area of the space 310 in which the support layer is not yet formed on the first substrate. Regarding the second substrate 200, the upper surface is not necessarily provided with specific functionality, but the lower surface needs to be more hydrophilic than the upper surface of the first substrate 100 to take advantage of the capillary or microfluidic structure. The volume and height of the space can be controlled by the support layer 300, and the upper surface of the first substrate 100, which is exposed in the space 310, needs to be more hydrophobic than the lower surface of the second substrate 200 to facilitate the unimpeded flow of the sample (solution).

The support layer 300 that defines the height of the space 310 is preferably made of a more hydrophobic material than the lower surface of the second substrate 200, and the space 310 can be formed through laser cutting. The sample inlet 10 which is an extension of the space can also be formed through laser cutting (see FIG. 1). The lower surface of the second substrate 200 is a film treated to be hydrophilic, and the gas outlet 20 may be formed through laser cutting. The shape of the gas outlet 20 of the second substrate 200 may be a circle, a square, or a rectangle but is not limited thereto.

The electrode layer 400 may be disposed on the first substrate 100. For example, the electrode layer 400 may be in contact with the upper surface of the first substrate 100. The electrode layer 400 may serve as a passage through which electrons or holes generated in an oxidation-reduction reaction of a detection target material are transferred.

In exemplary embodiments, the electrode layer 400 may be formed by printing a carbon paste film or a metal film on the first substrate 100 and patterning the carbon paste film or the metal film. Here, the metal film made of made of Au, Ag, Cu, Pt, Ti, Ni, Sn, Mo, Co, Pd, or an alloy of any combination thereof.

For the patterning, a patterning method commonly used in the art may be used. For example, photolithography may be used.

When the electrode layer 400 further includes a metal protective layer, the metal protective layer is formed after the metal film is patterned. Alternatively, a conductive oxide film made of indium tin oxide (ITO) or indium zinc oxide (IZO) is formed on the metal film, and the conductive oxide film and the metal film are patterned together to obtain the metal layer and the metal protective layer.

In exemplary embodiments, the electrode layer 400 may be formed of a single layer of carbon paste, a single layer of a mixture of carbon paste and a mediator, or a double layer including a carbon paste layer and an electroplating mediator layer formed on the carbon paste layer. Since the carbon paste layer is provided as an electrode, an additional metal electrode may not be required. Thus, the thickness of the biosensor can be reduced. In addition, the types of the mediator may include at least one type selected from the group consisting of potassium ferricyanide, cytochrome C, pyrroroquinoline quinone (PQQ), NAD+, NADP+, copper complexes, ruthenium compounds, phenazine methosulfate, phenazine methosulfate derivatives (PMS), potassium ferricyanide (K₃[Fe(CN)₆]), potassium ferrocyanide (K₄[Fe(CN)]₆), hexaamineruthenium (III) chloride, ferrocene, ferrocene derivatives, quinones, quinone derivatives, and hydroquinone.

The electrode layer 400 may be composed of a working electrode layer 410 and a reference electrode layer 420. Alternatively, the electrode layer 1400 may further include a wiring unit 430 electrically connected to the working electrode layer 410 and/or the reference electrode layer 420.

The working electrode layer 410 is a component to sense an electrical signal generated by a reaction of an analyte included in a sample. In one embodiment, an enzyme reaction layer 500 may be formed on the working electrode layer 410, and a polymer membrane layer (not shown) may be further formed on the enzyme reaction layer 500.

The working electrode layer 410 may be disposed on the first substrate 100. In one embodiment, the working electrode layer 410 may be disposed in contact with the upper surface of the first substrate 100. The working electrode layer 410 may serve as a passage through which electrons or holes generated in an oxidation-reduction reaction of a sensing target material are transferred. Specifically, the working electrode layer 410 can detect an electrical signal generated by a reaction between an enzyme in the enzyme reaction layer 500 and the sensing target material. The sensing target material may be, but is not limited to, human sweat, bodily fluid, blood, and the like. For example, the sensing target material may be glucose or lactic acid (lactate).

In one or more embodiments, the working electrode layer 410 may include one or more selected from the group consisting of a carbon electrode layer and a metal electrode layer.

In one or more embodiments, the carbon electrode layer contains at least one type selected from the group consisting of carbon paste, pyrolytic graphite, glassy carbon, perfluorocarbon (PFC), carbon nanotubes (CNTs), and the like. The carbon electrode layer can stably transport electrons and/or holes generated in the enzyme reaction layer 500.

In one or more embodiments, the carbon electrode layer may be a single layer with or without a mediator, and may further include a mediator layer formed by electroplating on a carbon electrode layer. For example, the mediator may include at least one type selected from the group consisting of potassium ferricyanide, cytochrome C, pyrroroquinoline quinone (PQQ), NAD+, NADP+, copper complexes, ruthenium compounds, phenazine methosulfate, phenazine methosulfate derivatives (PMS), potassium ferricyanide (K₃[Fe(CN)₆]), potassium ferrocyanide (K₄[Fe(CN)]₆), hexaamineruthenium (III) chloride, ferrocene, ferrocene derivatives, quinones, quinone derivatives, and hydroquinone.

In one embodiment, the working electrode layer 410 may be formed as a single layer of carbon paste. Since the carbon paste single layer is provided as the electrode, the metal electrode may be omitted. Therefore, the thinning of the biosensor can be achieved.

In one embodiment, the metal electrode layer may be composed of a metal layer and a metal protective layer disposed on the metal layer.

In one or more embodiments, the metal layer contains at least one selected from the group consisting of gold (Au), silver (Ag), copper (Cu), platinum (Pt), titanium (Ti), nickel (Ni), tin (Sn), molybdenum (Mo), palladium (Pd), cobalt (Co), and alloys thereof. For example, an Ag—Pd—Cu alloy (APC alloy) may be used.

The metal protective layer has electrical conductivity and may cover the entire top surface of the metal layer. In one embodiment, the metal protective layer may be disposed in contact with the upper surface of the metal layer. The metal protective layer is to prevent the metal layer from being oxidized and/or reduced by the oxidation-reduction reaction of the working electrode layer 410.

In one or more embodiments, the working electrode layer 410 may be made of one type selected from the group consisting of indium tin oxide (ITO) and indium zinc oxide (IZO). TO and IZO have electrical conductivity and are chemically stable, thereby effectively preventing oxidation-reduction reactions of the metal layer. In addition, the metal protective layer prevents the direct contact of the metal layer with air, thereby preventing oxidation of metal elements constituting the metal layer. Accordingly, reliability of electrical signal sensing of the metal layer can be improved.

In one embodiment, the metal electrode layer may be provided between the first substrate and the carbon electrode layer.

The reference electrode layer 420 has a constant potential and serves as a reference electrode for obtaining a potential difference with the working electrode layer 410.

In one or more embodiments, the reference electrode layer 420 may include one or more kinds selected from the group consisting of a silver-silver chloride (Ag/AgCl) electrode, a calomel electrode, a mercury-mercury sulfate electrode, and a mercury-mercury-oxide mercury. In addition, the reference electrode layer 420 is preferably a silver-silver chloride (Ag/AgCl) electrode in terms that the hysteresis of the potential with respect to the temperature cycle is not significant, and the potential is stable over a temperature range up to a high temperature. A silver-silver chloride (Ag/AgCl) electrode may be formed from an Ag/AgCl paste.

The wiring unit 430 may be formed on the first substrate 100. In one embodiment, the wiring unit 430 may be disposed in contact with the upper surface of the first substrate 100, and may be electrically connected to the working electrode layer 410 and/or the reference electrode layer 420. The wiring unit 430 may be employed to serve as a channel for transmitting electrical signals such as signals measured from the working electrode layer 410 and the reference electrode layer 420 and driving signals.

In one embodiment, a wire connected to the working electrode layer 410 and a wire connected to the reference electrode layer 420 may be electrically isolated from each other.

In one embodiment, the wiring unit 430 may be formed of the same material as at least a portion of the working electrode layer 410 and/or the reference electrode layer 420. In some embodiments, the wiring unit 430 may be integrally formed with at least a portion of the working electrode layer 410 and/or the reference electrode layer 420. For example, the wiring unit 430 may be integrally formed by forming a metal film on the first substrate 100 and patterning the metal film.

The enzyme reaction layer 500 may be employed as a layer in which a chemical reaction of an analyte included in the sample occurs. In one embodiment, the enzyme reaction layer 500 may include an enzyme, a mediator, and a buffer.

The enzyme reaction layer 500 may be disposed on the electrode layer 400. For example, it may be in direct contact with the upper surface. Specifically, the enzyme reaction layer 500 may be disposed on the working electrode layer 410. In one embodiment, the enzyme reaction layer 500 may be disposed in contact with the upper surface of the working electrode layer 410.

According to one embodiment of the present application, the enzyme reaction layer may have an area (x) that is 60% to 330% with respect to the area of the working electrode layer 410, and preferably have an area (x) of 95% to 330% with respect to the area of the working electrode layer 410. For example, when the area of the upper surface of the working electrode layer 410 is about 2.83 mm², the area (x) of the enzyme reaction layer 500 may be about 1.7 to 9.4 mm² and preferably about 2.7 to 9.4 mm². When the above range is satisfied, the analysis resolution of the sample can be optimized. Specifically, when the area of the enzyme reaction layer is excessively small, measurement accuracy may decrease, whereas when the area is excessively large, measurement efficiency may decrease.

The enzyme may combine with an analyte contained in the sample to form an enzyme-substrate complex which adjusts the activation energy of a chemical reaction, thereby increasing or decreasing the speed of metabolism.

In one or more embodiments, the enzyme may be selected according to the type of a test material to be sensed, and may include one or more selected from the group consisting of oxidases and dehydrogenases.

In exemplary embodiments, the oxidase may include at least one of glucose oxidase, cholesterol oxidase, lactate oxidase, ascorbic acid oxidase, and alcohol oxidase, and the dehydrogenase may include at least one of glucose dehydrogenase, glutamate dehydrogenase, lactate dehydrogenase, and alcohol dehydrogenase.

The detection target analyte which can be measured may vary depending on the oxidase or dehydrogenase, and it may be glucose, lactate, cholesterol, ascorbic acid, alcohol, glutamic acid, and the like. The concentrations of the materials can be measured.

For example, when the biosensor is a lactate sensor, the enzyme reaction layer may contain lactate oxidase or lactate dehydrogenase.

In one embodiment, the oxidase or dehydrogenase may be cross-linked, immobilized, and cemented by a cross-linking agent. Examples of the crosslinking agent include a crosslinking agent commonly used in the art to which the present invention pertains, and in one embodiment, glutaraldehyde (GA) or chitosan may be used.

The enzyme reaction layer 500 may be formed by applying, for example, a composition obtained by mixing an oxidase or a dehydrogenase with a cross-linking agent onto the enzyme reaction layer and then drying the composition.

For the coating, a coating method commonly used in the art may be used, and for example, various printing methods such as drop casting may be used.

The enzyme reaction layer 500 may contain an enzyme having a Michaelis constant (Km) value of 0.01 to 10 mM. Therefore, it is possible to more effectively control the measurable concentration range of the sample determined by the Km value of the enzyme.

In addition, as the area of the enzyme reaction layer 500 increases, the amount of the reactant increases, and the measurable value increases. However, when the enzyme area is smaller than the area of the electrode layer 400, an error may occur in the measured value, and thus the dispersion also increases. On the other hand, when the enzyme area is excessively large compared to the area of the electrode layer 400, the required amount of the sample for analysis also increases, and thus it may be difficult to achieve the goal of providing a highly accurate biosensor that can analyze a very small amount of sample. In addition, it may be difficult to employ the cover composed of the support layer and the second substrate in the biosensor, the capillary effect may be deteriorated due to the excessively wide cover, or an analysis resolution may become lower than a required slope value. Therefore, the area of the enzyme reaction layer 500 is preferably 0.6 to 3.5 times, preferably 0.95 to 3.3 times, and more preferably 1.02 to 2.9 times the area of the working electrode layer 410 exposed to the sample.

The mediator may include at least one type selected from the group consisting of potassium ferricyanide, cytochrome C, pyrroroquinoline quinone (PQQ), NAD+, NADP+, copper complexes, ruthenium compounds, phenazinemethosulfate, phenazinemethosulfate derivatives (PMS), potassium ferricyanide (K₃[Fe(CN)₆]), potassium ferrocyanide (K₄[Fe(CN)₆]), hexaammineruthenium (III) chloride, ferrocene, ferrocene derivatives, quinones, quinone derivatives, and hydroquinones. Preferably, the mediate is a ruthenium compound, phenazine methosulfate, and a phenazine methosulfate derivative.

As the ruthenium compound, an existing ruthenium compound or a new ruthenium compound to be developed in the future may be used. The ruthenium compound is preferably one that can exist in the reaction system as an oxidized ruthenium complex. As long as the ruthenium complex functions as a mediator (electron transporter), the type of ligand is not particularly limited.

As the phenazine methosulfate and its derivatives, conventional or to-be-newly-developed compounds may be used. Examples thereof include phenazine methosulfate and 1-methoxy-5-methylphenazinium methyl sulfate (1-methoxy PMS).

The buffer may be appropriately selected according to the type and concentration of the detection target material (sample), and is not particularly limited as long as it can exhibit predetermined detection results even for samples having various pH values.

In one or more embodiments, the buffer includes one or more types selected from the group consisting of phosphate buffered saline (PBS), tris(hydroxymethyl) aminomethane (Tris), tris hydrochloric acid (Tris-HCl), ammonium bicarbonate, 4-(2-hydroxyethyl)-1-piperazineethanesulfonic acid (HEPES), 3-(N-morpholino)propanesulfonic acid (MOPS), 2-(N-morpholino)ethanesulfonic acid (MES), 2,2-bis(hydroxymethyl)-2,2′,2″-nitrilotriethanol (Bis-tris), N-(2-acetamido)iminodiacetic acid (ADA), piperazine-N,N′-bis(2-ethanesulfonic acid) (PIPES), N-(2-acetamido)-2-aminoethanesulfonic acid (ACES), 3-(N-morpholinyl)-2-hydroxypropanesulfonic acid sodium salt (MOPSO), 1,3-bis(tris (hydroxymethyl) methylamino)propane (Bis-tris propane), N,N-bis(2-hydroxyethyl)-2-aminoethanesulfonic acid (BES), 2-[1,3-dihydroxy-2-(hydroxymethyl)propan-2-yl]amino]ethanesulfonic acid (TES), 3-(bis(2-hydroxyethyl)amino)-2-hydroxypropane-1-sulfonic acid (DIPSO), 3-[1,3-dihydroxy-2-(hydroxymethyl)propan-2-yl]amino]-2-hydroxypropane-1-sulfonic acid (TAPSO), Trizma, piperazine-1,4-bis(2-hydroxypropanesulfonic acid)dihydrate (POPSO), 3-[4-(2-hydroxyethyl)-1-piperazinyl] propanesulfonic acid (HEPPS), N-(2-hydroxy-1,1-bis(hydroxymethyl)ethyl)glycine (TRICINE), glycylglycine (GLY-GLY), 2-(bis(2-hydroxyethyl)amino)acetic acid (BICINE), N-(2-hydroxyethyl)piperazine-N′-(4-butanesulfonic acid) (HEPBS), 3-[1,3-dihydroxy-2-(hydroxymethyl)propan-2-yl]amino]propane-1-sulfonic acid (TAPS), 2-amino-2-methyl-1,3-propanediol (AMPD), N-(1,1-dimethyl-2-hydroxyethyl)-3-amino-2-hydroxypropanesulfonic acid (AMPSO), N-cyclohexyl-2-aminoethanesulfonic acid (CHES), N-cyclohexyl-2-hydroxyl-3-aminopropanesulfonic acid (CAPSO), 1-amino-2-methyl-1-propanol (AMP), N-cyclohexyl-3-aminopropanesulfonic acid (CAPS), 4-(cyclohexylamino)-1-butanesulfonic acid (CABS), and Lysogeny broth.

In one embodiment, the enzyme reaction layer 500 may further contain deionized water (DI water) as a solvent for mixing the above-described components.

The detection principle using the enzyme reaction layer 500 will be described below. For example, when a sample containing an analyte is injected into the biosensor, the analyte (for sample, substrate) included in the sample is oxidized by oxidase or dehydrogenase, and the oxidase or dehydrogenase is reduced. In this case, the electron transfer mediator rapidly induces the reaction of the enzyme by causing a catalytic reaction, oxidizes the oxidase or dehydrogenase, and is reduced. The reduced electron transfer mediator loses electrons on the electrode surface to which a certain voltage is applied and is electrochemically oxidized back. Since the concentration of the analyte in the sample is proportional to the amount or density of the current generated in the process of oxidation of the electron transfer mediator, the concentration of the analyte can be determined by measuring the amount or density of the current.

The polymer membrane layer (not illustrated) may be disposed on the enzyme reaction layer 500. In one embodiment, the polymer membrane layer may be disposed in contact with the upper surface of the enzyme reaction layer 500. The polymer membrane layer is a selective permeable membrane. The polymer membrane layer serves as a layer for improving the detection performance of the biosensor by preventing oxidation of the enzyme and and protecting the enzyme against foreign substances, thereby increasing stability of the enzyme.

In one or more embodiments, the polymer membrane layer may contain one or more polymers selected from the group consisting of fluorine-based polymers, water-soluble polymers, and water-insoluble polymers.

In one or more embodiments, the fluorine-based polymer may include a perfluoro-based polymer such as a perfluorosulfonic acid-based resin, and may include, for example, Nafion® (registered trademark) of DuPont.

In one or more embodiments, the water-soluble polymer may include one or more types selected from the group consisting of polyvinyl alcohol (PVA), hydroxyethyl cellulose (HEC), hydroxypropyl cellulose (HPC), carboxymethyl cellulose (CMC), cellulose acetate (CA), and polyvinyl pyrrolidone (PVP).

In one or more embodiments, the water-insoluble polymer may include at least one selected from the group consisting of polyurethane (PU), polycarbonate (PC), and polyvinyl chloride (PVC).

### <Method of Manufacturing Biosensor>

The present disclosure includes a biosensor manufacturing method for manufacturing the biosensor described above.

In one embodiment, a working electrode may be manufactured by forming a working electrode layer on a first substrate, forming an enzyme reaction layer on the working electrode layer, and forming a polymer membrane layer on the enzyme reaction layer.

In one or more embodiments, the forming of the working electrode layer may be performed by one or more processes selected from the group consisting of screen printing, letterpress printing, intaglio printing, lithography, and photolithography.

For example, a carbon paste is printed on a substrate by screen printing, a metal film containing one or more selected from the group consisting of gold (Au), silver (Ag), copper (Cu), platinum (Pt), titanium (Ti), nickel (Ni), tin (Sn), molybdenum (Mo), palladium (Pd), cobalt (Co), and alloys thereof, and the carbon paste and the metal film are patterned through a photolithography method or the like.

In one embodiment, when the working electrode layer further includes a metal protective layer, the metal protective layer is formed after the metal layer is first pattern. Alternatively, after a conductive oxide film of indium tin oxide (ITO) or indium zinc oxide (IZO) is formed on the metal film, and the metal film and the conductive oxide film are then patterned together to form the metal layer and the metal protective layer.

The forming of the enzyme reaction layer and the forming of the polymer membrane layer may be formed by a coating method commonly used in the art to which the present invention pertains. In one or more embodiments, the coating method may be any one selected from the group consisting of flow coating, ink jet, and drop casting, and preferably, the coating method may be drop casting.

In one embodiment, the reference electrode may be formed using Ag/AgCl paste or the like, and may be formed by substantially the same method as the method of forming the working electrode.

In one embodiment, the biosensor may be manufactured by first bonding the support layer 300 and the second substrate 200 with a lamination machine, and then bonding the electrode layer 400 thereto. In another embodiment, the biosensor may be manufactured by first bonding the first substrate 100 including the electrode layer 400 to the support layer 300 and then bonding the second substrate 200 thereto using a lamination machine.

A biosensor manufactured by the above-described manufacturing method may exhibit all of the characteristics that have been described above.

### <Method of Measuring Biosensor Signal>

The present invention includes a method for measuring an electrochemical signal of an analyte using the biosensor manufactured by the biosensor manufacturing method described above.

In the present specification, “electrochemical measurement” refers to measurement by applying an electrochemical measurement method, and in one or more embodiments, an amperometric method, a potentiometric method, a coulometric method, etc. may be used, and preferably, an amperometric method is used.

In one embodiment, the method for measuring a biosensor signal of the present disclosure includes: applying a voltage to an electrode unit including the working electrode and the reference electrode after brining a sample into contact with the biosensor; measuring a response current value generated upon application, and calculating an electrochemical signal of the analyte contained in the sample on the basis of the response current value. The applied voltage is not particularly limited, but in one or more embodiments, the voltage may be −500 to +500 mV and preferably −200 to +200 mV when using the silver-silver chloride electrode (Ag/AgCl electrode).

In one embodiment, in the biosensor signal measurement method of the present disclosure, a voltage may be applied to the electrode unit after a predetermined resting period during which the voltage is not applied after contact with the sample. Alternatively, the contact with the sample and the voltage application may be performed simultaneously.

The biosensor signal measurement method of the present disclosure can improve the accuracy and precision of the biosensor by minimizing the fluctuations in measurements for samples with different pH values, thereby further improving the sensitivity of the biosensor.

### <Biosensor Signal Measurement System>

The present disclosure relates to a biosensor electrochemical signal measurement system for measuring an electrochemical signal of an analyte contained in a sample, in which the system includes the biosensor, a voltage application unit that applies a voltage to the electrode unit of the biosensor, and a current measurement unit that measures a current in the electrode unit.

The biosensor signal measurement system of the present disclosure exhibits high measurement accuracy and precision even for samples showing various pH ranges and can improve the measurement sensitivity.

The voltage application unit is not particularly limited as long as it can form an electrical conduction path to the electrode unit of the biosensor and can apply a voltage. A known voltage application means can be used as the voltage application unit. In one or more embodiments, the voltage application unit may include a contactor capable of making contact with the electrode unit of the biosensor, and a power supply such as a DC power supply.

The measurement unit measures a plurality of electrical currents generated in the electrode unit when the voltage is applied. In one or more embodiments, the measurement unit may be any current measuring means that can measure a response current voltage corresponding to the amount of electrons emitted from the electrode unit of the biosensor. Any conventional current measuring means or any novel current measuring means to be developed can be used.

### Mode for Invention

Hereinafter, specific examples of the present disclosure will be described. The present disclosure may, however, be embodied in many different forms and should not be construed as being limited to the embodiments or examples described herein. Rather, these embodiments or examples are provided so that the present disclosure will be thorough and complete and will fully convey the concept of the invention to those skilled in the art. Thus, the present disclosure will be defined only by the scope of the appended claims.

### PREPARATION EXAMPLE

A bi-electrode structure composed of a working electrode and a reference electrode was provided on a first substrate (180 μm, PET) having a surface that is relatively hydrophobic. The working electrode and the reference electrode each had a size of 2.83 mm² and were fabricated by screen-printing a carbon paste and Ag/AgCl on the first substrate.

A certain amount of glucose oxidase was applied to the working electrode by hand dropping or using a dispenser, and then a certain amount of chitosan was applied in the same manner. After the process was completed, a certain amount of Nafion was applied in the same manner. Afterwards, a certain amount of glutaraldehyde was applied in the same manner. Next, after 1 to 3 minutes of waiting, washing was performed with PBS, followed by complete drying to form an enzyme reaction layer.

A gas outlet was formed through laser cutting on a second substrate (ABF-AFG of AMTE) having a surface that is relatively hydrophilic compared to the first substrate. OCA (Korea 3M, 8146-4) was laminated on the second substrate using a lamination machine to form a support layer. A biosensor was manufactured by bonding the second substrate to which the OCA was laminated to the first substrate having the electrode formed thereon, using a lamination device.

In addition, the biosensors manufactured were divided into a reduced type, a basic type, and an expanded type that were named according to the width of the space defined by the support layer. FIG. 8 schematically illustrate the structure of each of the reduced type, the basic type, and the expanded type.

### Example 1: Manufacture of Biosensor with Support Layer with Thickness of 50 μm

In the preparation example, the height (thickness) of the support layer (OCA) was set to 50 μm so that the height of the space became 50 μm, and the biosensors of reduced type, basic type, and expanded type structures as shown in FIG. 8 were manufactured separately.

### Example 2: Manufacture of Biosensor with Support Layer with Thickness of 100 μm

In the preparation example, the height (thickness) of the support layer was set to 100 μm so that the height of the space became 100 μm, and the biosensors of reduced type, basic type, and expanded type structures as shown in FIG. 8 were manufactured separately.

### Example 3: Manufacture of Biosensor with Support Layer with Thickness of 150 μm

In the preparation example, the height (thickness) of the support layer was set to 150 μm so that the height of the space became 150 μm, and the biosensors of reduced type, basic type, and expanded type structures as shown in FIG. 8 were manufactured separately.

### Comparative Example 1: Manufacture of Spaceless Biosensor

In the preparation example, a biosensor having no support layer and no second substrate was manufactured.

### Comparative Example 2: Manufacture of Biosensor with Support Layer with Thickness of 25 μm

In the preparation example, the height (thickness) of the support layer was set to 25 μm so that the height of the space became 25 μm, and the biosensor of a basic type structure was manufactured.

### Experimental Example 1

The minimum requisite amounts of sample required for measurement according to the height and structure of the support layer were investigated, and the results are summarized in Table 1 below. The sample was a body fluid.

TABLE 1

| | Comparative | | | | | | | | | | |
|---|---|---|---|---|---|---|---|---|---|---|---|
| | Example | | Example | | | | | | | | |
| Type | 1 | 2 | 1 | | | 2 | | | 3 | | |
| Structure* | — | a | a | b | c | a | b | c | a | b | c |
| Sample (μL) | ≥20 | 0.76 | 1.5 | 0.95 | 2.8 | 3 | 1.9 | 5.6 | 4.5 | 2.8 | 8.4 |

*a = basic type structure, b = reduced type structure, c = expanded type structure (see FIG. 8).

In addition, on the basis of the results of Table 1, the slopes according to the thicknesses of the support layer are shown in FIG. 11.

As can be seen from Table 1, in the case of Comparative Example 1, a relatively large amount of sample was required for glucose measurement. Therefore, the structure of the Comparative Example 1 in which a cover was not formed was not suitable for measurement of a body fluid sample that is difficult to collect in a large amount.

In addition, in the case of the reduced type structure of Example 1, the amount of sample was excessively small, for example, less than 1 μL, and in the case of the expanded type structure of Examples 2 and 3, the amount of sample exceeded 5 μL. In Experimental Example 2 below, the analysis resolution was investigated for the case where the sample amount satisfies the range of 1 to 5 μL.

### Experimental Example 2

In order to investigate the slope influence factor, the analysis resolution change according to the height and structure of the support layer was checked for Examples 1 to 3 in which measurement can be performed a small amount of sample. The results are summarized in Table 2 below.

Specifically, the resolution is obtained by measuring the current for a sample using a CHI630 instrument and converting the concentration of the sample and the current value to a slope (nA/mM). In the present experimental example, the biosensors of the examples and the comparative were manufactured such that the area of the enzyme reaction layer was 140% with respect to the area (2.83 mm²) of the reference electrode.

TABLE 2

| | Comparative | Example | | | | | |
|---|---|---|---|---|---|---|---|
| Type | Example 2 | 1 | | 2 | | 3 | |
| Structure* | a | a | c | a | b | a | b |
| Slope (nA/mM) | — | 61 | 52 | 213 | 234 | 354 | 359 |

*a = basic type structure, b = reduced type structure, c = expanded type structure (see FIG. 8).

In addition, on the basis of the results of Table 2, the slopes according to the heights of the support layer are indicated on the left side of FIG. 11, and the slopes according to the structures (solution volumes) are shown on the right side.

As shown in Table 2 and FIG. 11, in the case of the biosensor having the structure according to the Examples, it was confirmed that the resolution improved as the height of the support layer increased, but there was no significant correlation between the resolution and the solution volume.

On the other hand, in the case of Comparative Example 2, although the amount of sample measurable was confirmed to be small through Experimental Example 1, the thickness of the enzyme reaction layer was similar to the thickness of the space (height of the support layer), so the capillary effect was insufficient. In addition, the area of the enzyme reaction layer exposed to the sample to be measured varied from sample to sample, and thus the error range was large, and it was difficult to measure the slopes.

### Experimental Example 3

In this experimental example, for the structure confirmed to have better resolution between the basic type structure or the reduced type structure as in Examples 1 to 3 through Experimental Example 2, the change in resolution (slope) according to the area of the enzyme reaction layer was confirmed in the same manner as in Experimental Example 2, and the results are summarized in Table 3 and FIGS. 9 and 10.

TABLE 3

| Type | Example 1 | | | Example 2 | | | Example 3 | | |
|---|---|---|---|---|---|---|---|---|---|
| Structure* | a | | | b | | | b | | |
| Area of enzyme(mm²)** | A | B | C | A | B | C | A | B | C |
| Slope (nA/mM) | 40 | 61 | 70 | 159 | 234 | 263 | 260 | 359 | 377 |

*a = basic type structure, b = reduced type structure, c = expanded type structure (see FIG. 8),
**A = about 90% of the area of the working electrode, B = about 140% of the area of the working electrode, and C = about 210% of the area of the working electrode.

As can be seen from Table 3 and FIG. 10, in the structure according to the Examples, the resolution (slope) improved as the area of the enzyme reaction layer increased.

In addition, through the above experiments, it was confirmed that the resolution of the biosensor was greatly affected by the height of the support layer and the area of the enzyme reaction layer.

### INDUSTRIAL APPLICABILITY

The biosensor of the present disclosure can accurately measure glucose even with a very small amount of body fluid. Specifically, the biosensor of the present disclosure has a structure in which an empty space is formed by arranging the cover composed of the support layer and the upper substrate on the enzyme reaction layer. Thus, the biosensor of the present disclosure improves the resolution by adjusting the height of the support layer and the width of the enzyme reaction layer, thereby providing the effect of measuring finer concentrations even with a small amount of body fluid (sample).

1. A biosensor comprising:
a first substrate having a hydrophobic surface on one side;
a second substrate disposed on the first substrate and having a hydrophilic surface on one surface;
a support layer having a predetermined height to provide a space between the hydrophobic surface of the first substrate and the hydrophilic surface of the second substrate;
one or more electrode layers formed within the space on the first substrate; and
an enzyme reaction layer formed on the electrode layer.

2. The biosensor of claim 1, wherein the height of the support layer is larger than 25 μm and smaller than 300 μm.

3. The biosensor of claim 1, wherein the support layer is made of a polymer resin.

4. The biosensor of claim 1, wherein the enzyme reaction layer comprises at least one selected from the group consisting of oxidases and dehydrogenases.

5. The biosensor of claim 4, wherein the oxidases comprise at least one selected from the group consisting of glucose oxidase, cholesterol oxidase, lactate oxidase, ascorbic acid oxidase, and alcohol oxidase, and
the dehydrogenases comprise at least one selected from the group consisting of glucose dehydrogenase, glutamate dehydrogenase, lactate dehydrogenase, and alcohol dehydrogenase.

6. The biosensor of claim 1, wherein the electrode layer comprises a working electrode layer and a reference electrode layer.

7. The biosensor of claim 6, wherein the enzyme reaction layer is formed on the working electrode layer and has an area that is 60% to 330% times the area of the working electrode layer.

8. The biosensor of claim 1, wherein the electrode layer comprises at least one selected from the group consisting of a carbon electrode layer and a metal electrode layer.

9. The biosensor of claim 8, wherein the metal electrode layer comprises a metal layer and a metal protective layer formed on the metal layer.
